(19) United States
(12) Patent Application Publication
(10) Pub. No.: US 2024/0254216 A1
(43) Pub. Date: Aug. 1, 2024

(54) VARIANT LIBRARY-COUPLED IMMUNOGENICITY MAPPING OF MONOCLONAL ANTIBODY THERAPEUTICS

(71) Applicant: VANDERBILT UNIVERSITY, Nashville, TN (US)

(72) Inventors: Jeremy Goettel, Nashville, TN (US); Justin Jacobse, Nashville, TN (US); Ivelin Georgiev, Nashville, TN (US); Lauren Walker, Nashville, TN (US); Clinton Holt, Nashville, TN (US)

(21) Appl. No.: 18/428,118

(22) Filed: Jan. 31, 2024

Related U.S. Application Data
(60) Provisional application No. 63/482,367, filed on Jan. 31, 2023.

(51) Int. Cl. C07K 16/24; A61K 39/00; G01N 33/50; G01N 33/58; G01N 33/68

(52) U.S. Cl. CPC C07K 16/241; G01N 33/5052; G01N 33/58; G01N 33/6878; A61K 2039/505; C07K 2317/21; C07K 2317/76

(57) ABSTRACT

A method for generating deimmunized antibody therapeutics, including: labeling a plurality of mutated variants of an antibody therapeutic with unique barcodes; providing a plurality of barcode-labeled mutated variants to a population of antibody therapeutic-specific B-cells; allowing the plurality of barcode-labeled mutated variants to bind to the population of antibody therapeutic-specific B-cells; and identifying unbound mutated variants as having lower immunogenicity than the antibody therapeutic.

### CROSS-REFERENCE TO RELATED APPLICATIONS

This application claims priority to, and the benefit of, U.S. Provisional Patent Application No. 63/482,367, filed Jan. 31, 2023, which is incorporated by reference herein in its entirety.

### REFERENCE TO SEQUENCE LISTING

The sequence listing submitted on Jan. 31, 2024, as an .XML file entitled “10644-156US1_ST26.xml” created on Jan. 30, 2024, and having a file size of 3,956 bytes is hereby incorporated by reference pursuant to 37 C.F.R. § 1.52(c)(5).

### BACKGROUND

For many inflammatory diseases, treatment with monoclonal antibodies (mAbs) that neutralize soluble pro-inflammatory proteins has been an effective therapeutic strategy. However, many patients who respond to treatment initially stop responding due to the development of anti-drug antibodies, known as secondary non-responders. Even though fully humanized antibodies such as adalimumab (anti-tumor necrosis factor alpha (TNF)) have been developed, the administration of mAbs can result in the formation of anti-drug antibodies. Thus, anti-drug antibodies are a major clinical problem for mAb drugs.

A meta-analysis assessing the rates of immunogenicity of biologics found the development of anti-drug antibodies to adalimumab to be as high as 38%, with 20% of patients shown to develop anti-drug IgG antibodies at a median of 34 weeks. Limited studies in patients with rheumatoid arthritis (RA) who developed anti-adalimumab antibodies documented that the anti-drug antibodies are directed towards the TNF binding region of adalimumab, impeding TNF neutralization and it is likely that a limited number of epitopes are responsible for the anti-drug antibody responses to mAbs. Importantly, the anti-adalimumab response in patients with rheumatoid arthritis (RA) revealed that the anti-drug response was not due to allotypic mismatch, highlighting the need to focus on the fragment antigen-binding (Fab) portion of the drug. However, the low-throughput nature of these studies precludes mapping immunodominant epitopes for which BCRs are directed, rendering the full epitope repertoire of anti-adalimumab antibodies unknown.

Anti-drug antibodies are produced by differentiated B cells (plasmablasts) and terminally differentiated B cells (plasma cells) following V(D)J rearrangement of their B cell receptor (BCR), resulting in a diverse pool of antibody-producing B cells specific for antigens with high affinity. Although this process allows for the development of a vast antibody repertoire against pathogens, it can be problematic for mAbs therapies. Thus, it is critical to identify the epitopes that anti-drug antibodies target to effectively modify mAbs to be immune-tolerant. To date, predicting or defining the immunogenicity of mAbs has been challenging and generally limited to in silico and low-throughput methods.

### SUMMARY

In one aspect, disclosed herein is a method for generating deimmunized antibody therapeutics, comprising: labeling a plurality of mutated variants of an antibody therapeutic with unique barcodes; providing a plurality of barcode-labeled mutated variants to a population of antibody therapeutic-specific B-cells; allowing the plurality of barcode-labeled mutated variants to bind to the population of antibody therapeutic-specific B-cells; and identifying unbound mutated variants as having lower immunogenicity than the antibody therapeutic.

In another aspect, provided is a system for generating deimmunized antibody therapeutics, comprising: a plurality of barcode-labeled mutated variants of an antibody therapeutic; a population of antibody therapeutic-specific B-cells; and wherein the mutated variants are identified by a non-transitory computer-readable medium having instructions stored thereon, wherein the instructions, when executed by a processor, cause the processor to: determine mutations and/or combinations of mutations to the antibody therapeutic which do not substantially interfere with binding of the mutated variant to a target antigen.

In yet another aspect, disclosed herein is a mutated variant of adalimumab, comprising one or more mutations to SEQ ID NO: 1 and/or one or more mutations to SEQ ID NO: 2; wherein one or more mutations to SEQ ID NO: 1 are selected from the group consisting of D62K, Y101H, T28H, N54H, N54K, D31Q, S55G, S103W, S103H, W53K, G56Y, L102K, and H57W; or wherein the one or more mutations to SEQ ID NO: 2 are selected from the group consisting of Q27R, T69R, Q27W, A50N, S60Y, N31R, S67E, S67W, A94Y, N92G, and N31H.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF DRAWINGS

FIG. 1 depicts an example method of VAriant Library-coupled Immunogenicity Mapping of monoclonal Antibody therapeutics through antigen-specific B cell receptor sequencing (ValimAb-seq).

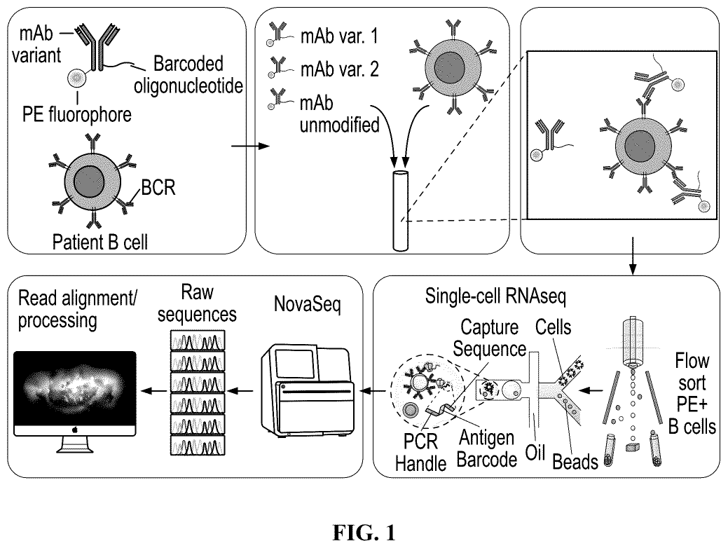

FIG. 2 depicts the quantification of TNF binding affinity for adalimumab and adalimumab variants. Adalimumab and variants were assessed for binding to recombinant human TNF using ELISA.

FIGS. 3A-3C depict TNF neutralization for adalimumab and adalimumab variants. FIG. 3A shows adalimumab and variants were assessed for neutralization TNF in vitro by stimulating AGS reporter cells in the presence of variants. FIG. 3B shows a correlation (Pearson correlation coefficient) for TNF binding affinity (FIG. 2) and TNF neutralization (FIG. 3A). FIG. 3C shows additional neutralization data.

FIGS. 4A-4B depict plasma levels in the patient cohort. FIG. 4A shows adalimumab plasma levels at inclusion and FIG. 4B shows anti-adalimumab plasma levels at inclusion, as determined by Sanquin.

FIGS. 5A-5D depict flow cytometry of antigen (adalimumab)-specific B cells. FIG. 5A shows flow cytometry of lymphocytes from a healthy control (left) and patient with anti-adalimumab antibodies. FIG. 5B shows flow cytometry of TNF on monocytes from a patient with anti-adalimumab antibodies. FIG. 5C shows flow cytometry of TNF on B cells from a patient with anti-adalimumab antibodies. FIG. 5D shows flow cytometry of lymphocytes from a patient with anti-adalimumab antibodies before (top) and after (bottom) CD19 enrichment using negative selection. Cells were stained with adalimumab and variants.

### DETAILED DESCRIPTION

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

### Terminology

In this specification and in the claims that follow, reference will be made to a number of terms, which shall be defined to have the following meanings:

Throughout the description and claims of this specification, the word “comprise” and other forms of the word, such as “comprising” and “comprises,” means including but not limited to, and are not intended to exclude, for example, other additives, segments, integers, or steps. Furthermore, it is to be understood that the terms comprise, comprising, and comprises as they relate to various aspects, elements, and features of the disclosed invention also include the more limited aspects of “consisting essentially of” and “consisting of.”

As used herein, the singular forms “a,” “an,” and “the” include plural referents unless the context clearly dictates otherwise. Thus, for example, reference to a “cell” includes aspects having two or more such cells unless the context clearly indicates otherwise.

Ranges can be expressed herein as from “about” one particular value and/or to “about” another particular value. When such a range is expressed, another aspect includes from the one particular value and/or to the other particular value. Similarly, when values are expressed as approximations, by use of the antecedent “about,” it will be understood that the particular value forms another aspect. It should be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

For the terms “for example” and “such as,” and grammatical equivalences thereof, the phrase “and without limitation” is understood to follow unless explicitly stated otherwise.

“Nucleotide,” “nucleoside,” “nucleotide residue,” and “nucleoside residue,” as used herein, can mean a deoxyribonucleotide, ribonucleotide residue, or another similar nucleoside analogue. A nucleotide is a molecule that contains a base moiety, a sugar moiety and a phosphate moiety. Nucleotides can be linked together through their phosphate moieties and sugar moieties creating an internucleoside linkage.

The method and the system disclosed here including the use of primers, which are capable of interacting with the disclosed nucleic acids, such as the antigen barcode as disclosed herein. In certain embodiments the primers are used to support DNA amplification reactions. Typically, the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically, the disclosed primers hybridize with the disclosed nucleic acids or region of the nucleic acids or they hybridize with the complement of the nucleic acids or complement of a region of the nucleic acids.

The term “amplification” refers to the production of one or more copies of a genetic fragment or target sequence, specifically the “amplicon”. As it refers to the product of an amplification reaction, amplicon is used interchangeably with common laboratory terms, such as “PCR product.”

The term “polypeptide” refers to a compound made up of a single chain of D- or L-amino acids or a mixture of D- and L-amino acids joined by peptide bonds.

As used herein, the term “antigen” refers to a molecule that is capable of binding to an antibody. In some embodiment, the antigen stimulates an immune response such as by production of antibodies specific for the antigen.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

Each antibody molecule is made up of the protein products of two genes: heavy-chain gene and light-chain gene. The heavy-chain gene is constructed through somatic recombination of V, D, and J gene segments. In humans, there are 51 VH, 27 DH, 6 JH, 9 CH gene segments on human chromosome 14. The light-chain gene is constructed through somatic recombination of V and J gene segments. There are 40 Vκ, 31 Vλ, 5 Jκ, 4 Jλ gene segments on human chromosome 14 (80 VJ). The heavy-chain constant domains that correspond to the different classes of immunoglobulins are called α, δ, ε, γ, and μ, respectively. The “light chains” of antibodies from any vertebrate species can be assigned to one of two clearly distinct types, called kappa (κ) and lambda (λ), based on the amino acid sequences of their constant domains.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or antigen binding fragment thereof” or “antibody or fragments thereof” encompasses chimeric antibodies and hybrid antibodies, with dual or multiple antigen or epitope specificities, and fragments, such as F(ab′)2, Fab′, Fab, Fv, sFv, scFv and the like, including hybrid fragments. Thus, fragments of the antibodies that retain the ability to bind their specific antigens are provided. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or antigen binding fragment thereof” are conjugates of antibody fragments and antigen binding proteins (single chain antibodies). Also included within the meaning of “antibody or antigen binding fragment thereof” are immunoglobulin single variable domains, such as for example a nanobody.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

### Compositions, Antibodies, and Methods

In an aspect, disclosed herein is a method for generating deimmunized antibody therapeutics, including: labeling a plurality of mutated variants of an antibody therapeutic with unique barcodes; providing a plurality of barcode-labeled mutated variants to a population of antibody therapeutic-specific B-cells; allowing the plurality of barcode-labeled mutated variants to bind to the population of antibody therapeutic-specific B-cells; and identifying unbound mutated variants as having lower immunogenicity than the antibody therapeutic.

In some aspects, the method can further include: determining, based on mutations present in bound and unbound mutated variants, an epitope of the antibody therapeutic; and preparing one or more additional mutated variants having lower immunogenicity than the antibody therapeutic based on the epitope of the antibody therapeutic.

In some aspects, unbound mutated variants can be determined by: washing unbound mutated variants from the population of therapeutic-specific B-cells; separating the therapeutic-specific B-cells into single cell emulsions; introducing into each single cell emulsion a unique cell barcode-labeled bead; preparing a single cell cDNA library from the single cell emulsions; performing PCR amplification reactions to produce a plurality of amplicons, wherein the amplicons comprise: 1) the cell barcode and the mutated variant barcode, and 2) the cell barcode and i) an immunoglobulin heavy chain (VDJ) sequence, or ii) an immunoglobulin light chain (VJ) sequence; and sequencing the plurality of amplicons.

In other aspects, unbound mutated variants can be determined by: washing unbound mutated variants from the population of therapeutic-specific B-cells; optionally isolating mutated variant positive cells using fluorescence-activated cell sorting; separating the therapeutic-specific B-cells into single cell emulsions; introducing into each single cell emulsion a unique cell barcode-labeled bead; preparing a single cell cDNA library from the single cell emulsions; performing PCR amplification reactions to produce a plurality of amplicons, wherein the amplicons comprise: 1) the cell barcode and the mutated variant barcode, and 2) the cell barcode and i) an immunoglobulin heavy chain (VDJ) sequence, and/or ii) an immunoglobulin light chain (VJ) sequence; sequencing the plurality of amplicons; and identifying the i) mutated variant barcodes and ii) immunoglobulin heavy chain (VDJ) sequence and/or immunoglobulin light chain (VJ) sequence, with matching cell barcodes.

In yet other aspects, unbound mutated variants can be determined by any one of the methods disclosed in U.S. Pat. Pub. No. 2021/0302422 or U.S. Pat. Pub. No. 2022/0315982, each of which is hereby incorporated by reference in its entirety.

Also disclosed herein is a set of PCR primers for performing PCR amplification reactions to produce a plurality of amplicons, wherein the amplicons comprise: 1) the cell barcode and the antigen barcode, and 2) the cell barcode and i) an immunoglobulin heavy chain (VDJ) sequence, or ii) an immunoglobulin light chain (VJ) sequence.

As used herein, the term “beads” is not limited to a specific type of bead. Rather, a large number of beads are available and are known to one of ordinary skill in the art. A suitable bead may be selected on the basis of the desired end use and suitability for various protocols. In some embodiments, the bead is or comprises a particle or a bead. Beads can comprise particles that have been described in the art in, for example, U.S. Pat. Nos. 5,084,169, 5,079,155, U.S. Pat. No. 473,231, and U.S. Pat. No. 8,110,351, each of which is hereby incorporated by reference in its entirety. The particle or bead size can be optimized for binding a cell in a single-cell emulsion and optimized for the subsequent PCR reaction.

In some aspects, barcodes can include DNA sequences or RNA sequences. It should be understood that the barcodes described above are conjugated to the barcode-labeled mutated variant in a way that is known to one of ordinary skill in the art. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. An oligonucleotide barcode can also be conjugated to an antigen using the Solulink Protein-Oligonucleotide Conjugation Kit (TriLink cat no. S-9011) according to the manufacturer's instructions. Briefly, the oligo and protein are desalted, and then the amino-oligo is modified with the 4FB crosslinker, and the biotinylated antigen protein is modified with S-HyNic. Then, the 4FB-oligo and the HyNic-antigen are mixed together. This causes a stable bond to form between the protein and the oligonucleotide.

In some aspects, the antibody therapeutic can include atezolizumab, avelumab, sugemalimab, cosibelimab, durvalumab, avelumab, cemiplimab, camrelizumab, serplulimab, penpulimab, sintilimab, toripalimab, retifanlimab, dostarlimab, pembrolizumab, nivolumab, tremelimumab, ipilimumab, abciximab, alemtuzumab, belimumab, bemarituzumab, brentuximab, catumaxomab, cetuximab, necitumumab, panitumumab, cixutumumab, trastuzumab, margetuximab, pertuzumab, inotuzumab, moxetumomab, loncastuximab, rituximab, ibritumomab, tositumomab, ofatumumab, ublituximab, ocrelizumab, obinutuzumab, polatuzumab, Sacituzumab, enfortumab, belantamab, mirvetuximab, relatlimab, tisotumab, anifrolumab, odronextamab, epcoritamab, glofitamab, mosunetuzumab, talquetamab, teclistamab, tebentafusp, amivantamab, emicizumab, blinatumomab, adalimumab, infliximab, bevacizumab, alirocumab, rozanolixizumab, lebrikizumab, mirikizumab, faricimab, bimckizumab, tralokinumab, lebrikizumab, ustekinumab, Risankizumab, tildrakizumab, or any combination thereof. In some aspects, the antibody therapeutic can include adalimumab.

In some aspects, the population of antibody therapeutic-specific B-cells can include a memory B-cell, a plasma cell, a naïve B cell, an activated B-cell, or a B-cell line. In some aspects, the population of antibody therapeutic-specific B cells can be derived from a patient.

In some aspects, the mutated variant can include one or more point mutations.

In some aspects, the method can further include assessing binding to and/or neutralization of a target antigen by the mutated variants. In some aspects, the target antigen can include PD-L1, PD1, CTLA-4, CD41 (Integrin alpha-IIb), CD52, BAFF, FGFR2, CD 30 (TNFRSF8), CD3, EpCAM, EGFR, IGF-1 receptor(CD221), HER2, CD22, CD19, CD20, CD79b, Trop-2, Nectin-4, BCMA, Folate receptor alpha, LAG-3, Tissue factor, IFNAR1, G protein-coupled receptor 5D, BCMA, gp100, cMET, Factor IXa, Factor X, tumor necrotic factor alpha (TNFα), VEGF-A, PCSK9, FcRn, IL-13, IL-23p19, Ang-2, IL-17A,F, or any combination thereof. In some aspects, the target antigen can be tumor necrotic factor alpha (TNFα).

In some aspects, the method can further include preparing a modified antibody therapeutic comprising one or more mutated variants having lower immunogenicity than the antibody therapeutic. In some aspects, the method can further include administering the modified antibody therapeutic to a patient in need thereof. In some such aspects, when administered to the patient, the modified antibody therapeutic can generate less anti-drug antibodies than the antibody therapeutic without modifications.

In another aspect, disclosed herein is a system for generating deimmunized antibody therapeutics, including: a plurality of barcode-labeled mutated variants of an antibody therapeutic; a population of antibody therapeutic-specific B-cells. In some such aspects, the mutated variants can be identified by a non-transitory computer-readable medium having instructions stored thereon, wherein the instructions, when executed by a processor, can cause the processor to: determine mutations and/or combinations of mutations to the antibody therapeutic which do not substantially interfere with binding of the mutated variant to a target antigen. For example, and without limiting this disclosure to a single embodiment, the computer-readable medium may be configured to work in tandem with protein design platforms to determine appropriate mutations and/or combinations of mutations that avoid interfering with the binding. One non-limiting example of such a protein design platform is Osprey.

In some aspects, the instructions, when executed by the process, can further cause the process to determine mutations and/or combinations of mutations to the antibody therapeutic which minimize the possibility of interaction with the population of antibody therapeutic-specific B-cells.

In some aspects, the computer-readable medium can be a protein design platform configured to predict the structure and stability of mutant variants. Numerous protein design platforms can be utilized or configured according to embodiments of this disclosure. A suitable protein design platform to determine the structure and/or stability of a wildtype or mutated protein or peptide can reasonably be selected. Such a protein design platform can predict and model the structure of the antibody therapeutic and analyze the stability and energetics of said structure. Upon the introduction of one or more mutations, the platform can predict the resulting structure, stability, and/or other parameters of the mutated variant. The protein design platform can utilize, for example, scoring functions, machine learning, other suitable algorithms, and/or databases of existing proteins/peptides and mutations thereof to make such a prediction. The protein design platform can further suggest candidates based on criteria such as, but not limited to, stability, surface exposure, binding probability, or another suitable criterion.

In yet another aspect, disclosed is a mutated variant of adalimumab, including one or more mutations to SEQ ID NO: 1 and/or one or more mutations to SEQ ID NO: 1.

In some aspects, disclosed herein is a mutated variant of adalimumab, comprising one or more mutations to SEQ ID NO: 1 and/or one or more mutations to SEQ ID NO: 2; wherein one or more mutations to SEQ ID NO: 1 are selected from the group consisting of D62K, Y101H, T28H, N54H, N54K, D31Q, S55G, S103W, S103H, W53K, G56Y, L102K, and H57W; or wherein the one or more mutations to SEQ ID NO: 2 are selected from the group consisting of Q27R, T69R, Q27W, A50N, S60Y, N31R, S67E, S67W, A94Y, N92G, and N31H.

In one embodiment, the mutated variant of adalimumab comprises a D62K mutation, in comparison to SEQ ID NO: 1. In one embodiment, the mutated variant of adalimumab comprises a Y101H mutation, in comparison to SEQ ID NO: 1. In one embodiment, the mutated variant of adalimumab comprises a T28H mutation, in comparison to SEQ ID NO: 1. In one embodiment, the mutated variant of adalimumab comprises a N54H mutation, in comparison to SEQ ID NO: 1. In one embodiment, the mutated variant of adalimumab comprises a N54K mutation, in comparison to SEQ ID NO: 1. In one embodiment, the mutated variant of adalimumab comprises a D31Q mutation, in comparison to SEQ ID NO: 1. In one embodiment, the mutated variant of adalimumab comprises a S55G mutation, in comparison to SEQ ID NO: 1. In one embodiment, the mutated variant of adalimumab comprises a S103W mutation, in comparison to SEQ ID NO: 1. In one embodiment, the mutated variant of adalimumab comprises a S103H mutation, in comparison to SEQ ID NO: 1. In one embodiment, the mutated variant of adalimumab comprises a W53K mutation, in comparison to SEQ ID NO: 1. In one embodiment, the mutated variant of adalimumab comprises a G56Y mutation, in comparison to SEQ ID NO: 1. In one embodiment, the mutated variant of adalimumab comprises a L102K mutation, in comparison to SEQ ID NO: 1. In one embodiment, the mutated variant of adalimumab comprises a H57W mutation, in comparison to SEQ ID NO: 1.

In one embodiment, the mutated variant of adalimumab comprises a Q27R mutation, in comparison to SEQ ID NO: 2. In one embodiment, the mutated variant of adalimumab comprises a T69R mutation, in comparison to SEQ ID NO: 2. In one embodiment, the mutated variant of adalimumab comprises a Q27W mutation, in comparison to SEQ ID NO: 2. In one embodiment, the mutated variant of adalimumab comprises a A50N mutation, in comparison to SEQ ID NO: 2. In one embodiment, the mutated variant of adalimumab comprises a S60Y mutation, in comparison to SEQ ID NO: 2. In one embodiment, the mutated variant of adalimumab comprises a N31R mutation, in comparison to SEQ ID NO: 2. In one embodiment, the mutated variant of adalimumab comprises a S67E mutation, in comparison to SEQ ID NO: 2. In one embodiment, the mutated variant of adalimumab comprises a S67W mutation, in comparison to SEQ ID NO: 2. In one embodiment, the mutated variant of adalimumab comprises a A94Y mutation, in comparison to SEQ ID NO: 2. In one embodiment, the mutated variant of adalimumab comprises a N92G mutation, in comparison to SEQ ID NO: 2. In one embodiment, the mutated variant of adalimumab comprises a N31H mutation, in comparison to SEQ ID NO: 2.

In some aspects, the one or more mutations to SEQ ID NO: 1 can be selected from the group consisting of D62X, Y101X, T28X, N54X, D31X, S55X, S103X, W53X, G56X, L102X, and H57X, where X is any amino acid. In some such aspects, the one or more mutations to SEQ ID NO: 1 can be selected from the group consisting of D62K, Y101H, T28H, N54H, N54K, D31Q, S55G, S103W, S103H, W53K, G56Y, L102K, and H57W. In some aspects, the one or more mutations to SEQ ID NO: 2 can be selected from the group consisting of Q27X, T69X, A50X, S60X, N31X, S67X, A94X, N92X, and N31X, where X is any amino acid. In some such aspects, the one or more mutations to SEQ ID NO: 2 can be selected from the group consisting of Q27R, T69R, Q27W, A50N, S60Y, N31R, S67E, S67W, A94Y, N92G, and N31H.

In one embodiment, the mutated variant of adalimumab comprises a D62X mutation, in comparison to SEQ ID NO: 1, where X is any amino acid. In one embodiment, the mutated variant of adalimumab comprises a Y101X mutation, in comparison to SEQ ID NO: 1, where X is any amino acid. In one embodiment, the mutated variant of adalimumab comprises a T28X mutation, in comparison to SEQ ID NO: 1, where X is any amino acid. In one embodiment, the mutated variant of adalimumab comprises a N54X mutation, in comparison to SEQ ID NO: 1, where X is any amino acid. In one embodiment, the mutated variant of adalimumab comprises a D31X mutation, in comparison to SEQ ID NO: 1, where X is any amino acid. In one embodiment, the mutated variant of adalimumab comprises a S55X mutation, in comparison to SEQ ID NO: 1, where X is any amino acid. In one embodiment, the mutated variant of adalimumab comprises a S103X mutation, in comparison to SEQ ID NO: 1, where X is any amino acid. In one embodiment, the mutated variant of adalimumab comprises a W53X mutation, in comparison to SEQ ID NO: 1, where X is any amino acid. In one embodiment, the mutated variant of adalimumab comprises a G56X mutation, in comparison to SEQ ID NO: 1, where X is any amino acid. In one embodiment, the mutated variant of adalimumab comprises a L102X mutation, in comparison to SEQ ID NO: 1, where X is any amino acid. In one embodiment, the mutated variant of adalimumab comprises a H57X mutation, in comparison to SEQ ID NO: 1, where X is any amino acid.

In one embodiment, the mutated variant of adalimumab comprises a Q27X mutation, in comparison to SEQ ID NO: 2, where X is any amino acid. In one embodiment, the mutated variant of adalimumab comprises a T69X mutation, in comparison to SEQ ID NO: 2, where X is any amino acid. In one embodiment, the mutated variant of adalimumab comprises a A50X mutation, in comparison to SEQ ID NO: 2, where X is any amino acid. In one embodiment, the mutated variant of adalimumab comprises a S60X mutation, in comparison to SEQ ID NO: 2, where X is any amino acid. In one embodiment, the mutated variant of adalimumab comprises a N31X mutation, in comparison to SEQ ID NO: 2, where X is any amino acid. In one embodiment, the mutated variant of adalimumab comprises a S67X mutation, in comparison to SEQ ID NO: 2, where X is any amino acid. In one embodiment, the mutated variant of adalimumab comprises a S67X mutation, in comparison to SEQ ID NO: 2, where X is any amino acid. In one embodiment, the mutated variant of adalimumab comprises a A94X mutation, in comparison to SEQ ID NO: 2, where X is any amino acid. In one embodiment, the mutated variant of adalimumab comprises a N92X mutation, in comparison to SEQ ID NO: 2, where X is any amino acid. In one embodiment, the mutated variant of adalimumab comprises a N31X mutation, in comparison to SEQ ID NO: 2, where X is any amino acid.

### EXAMPLES

### Example 1. VAriant Library-Coupled Immunogenicity Mapping of Monoclonal Antibody Therapeutics Through Antigen-Specific B Cell Receptor Sequencing (ValimAb-Seq)

Despite decades of antibody discovery efforts, there is a paucity of data linking human antibody sequence to antigen specificity. One of the major reasons for such limited data is that even high-throughput antibody sequence identification methods, such as next-generation sequencing (NGS) of BCR sequences⁷, are decoupled from functional characterization. As a result, even though there are typically thousands to millions of antibody sequences within a single NGS dataset, functional information is obtained only for a handful of antibodies against a few antigens at a time. Other methods, such as antigen-specific B cell sorting or plate-based functional screening assays, have limited throughput, generating sequences for dozens to hundreds of antibodies, and are also restricted to targeting a specific antigen of interest.

ValimAb-seq technology involves physically mixing a B cell sample from a single patient with a library of DNA barcoded drug variants, thus transforming B cell-antigen binding into a sequenceable event. The major advantage of ValimAb-seq over capturing BCRs that interact with drugs across multiple patients is circumventing the exhaustive cloning of each unique BCR (some estimates ˜20-100 per patient) to identify the common immunogenic epitope(s).

Defining the immunogenic portion(s) of the therapeutic drug that anti-drug antibodies recognize will provide critical information to inform rational antibody design to mitigate immunogenicity while maintaining effective cytokine neutralization. A study was conducted which hypothesized that, by assessing the anti-drug antibody response to a therapeutic mAb that is shared across patients, it can be possible to find hotspots for anti-drug antibodies recognizing a mAb that then can be modified to yield a mAb therapeutic that is less immunogenic yet still efficacious. The study reports on developing a library of mAbs for the model drug adalimumab, which is then used to screen patient plasma for antibodies against adalimumab and efficacy in vitro, and efficacy and immunogenicity in murine models, and use this information to inform rational drug design. Importantly, the approach described herein for a model antigen is envisioned to be widely applicable to monoclonal antibodies.

### Methods

Variant design: OSPREY protein design software was used to select mutations on a high-resolution adalimumab-TNF structure (e.g., PDB id 3WD5)9. Briefly, the approach was as follows. First, the adalimumab structure was trimmed down to relevant regions in PyMOL. Then, point mutant targets were picked based on criteria of surface exposure, the possibility of anti-adalimumab antibody interaction, and how unlikely they are to disrupt antibody stability. Osprey was set up for energy calculations by treating the majority of the adalimumab—TNF alpha structure as rigid, but with backbone and side chain flexibility for residues in the near proximity of the residue being mutated. Finally, OSPREY algorithms were run to predict the best point mutation(s) based on both binding and energy favorability.

Variant generation: Genes were synthesized and cloned into proprietary heavy and light chain vectors by Twist. Vectors were transformed into competent DH5a E. coli cells and plated on ampicillin agar plates overnight. Colonies were selected from plated and cultured overnight in 400 mL. DNA was extracted using a maxi prep and sequenced by genewiz. The sequenced vectors were mapped back to the reference sequence using Geneious. Heavy and light genes were transfected into Expi293F cells and cultured for 4-5 days. After culture, the supernatant was filtered using a 0.22/0.45 μm PES filter, run over a protein A column, and the bound antibody from the protein A column eluted with 5 mL 100 mM glycine pH 2.7 into 1 M Tris-Cl, pH 8. The amount of protein was then measured using NanoDrop and 10 ug of the protein was after incubation for 5 minutes at 95° C. run on a SDS page gel in laemmli-only (non-reduced) or laemmli+beta-mercaptoethanol (reduced) for size confirmation (150 kDA full antibody, 50 kDa heavy chains, 25 kDA light chains). Finally, the antibody was run over a size exclusion column to remove any aggregates.

Antigen-Labeling: Variants were Labeled as Described Before.⁸

TNF binding: To assess variants binding to TNF, an ELISA plate was coated with 100 uL human recombinant TNF (750 ng mL-1) overnight at 4° C. After washing with PBS-T, the plate was blocked with 100 μL 5% milk in PBS-T for 1 hour at RT. After washing, 100 μL variants were incubated on the plate in 1% milk-PBS-T. After washing, an HRP-conjugated anti-IgG antibody was used at 1:10,000 in 1% milk-PBS-T. Following the final wash, detection was done using 100 μL TMB Blue ELISA substrate for approximately 10 minutes and terminated using 1 volume of IN sulfuric acid. OD was read at 450 nm.

TNF neutralization: Human adenogastric carcinoma cancer cells (AGS cells) transfected with an NF-KB luciferase reporter (9PIE849 pGL4.32[luc2P/NF—κB-RE/Hygro] from Promega, stably transfected cell line gift from Giovanni Suarez/Peck lab) were cultured in RPMI1640 with 10% HI FBS and 200 ng mL-1 hygromycin. Cells were plated at 40,000 cells/well in a flat bottom 96 well plate and incubated overnight. The next day, media was aspirated and 200 μL stimulation media was added. Stimulation media contained 100 ng mL-1 variants and 100 ng mL-1 human recombinant TNF and was incubated for 30 minutes before adding to the cells in an effort to let variants and TNF bind to each other. PMA (1 ug mL⁻¹) was used as a positive control during titration experiments. Cells were incubated for 4 hours followed by luciferase detection using the Luciferase Assay System with Reporter Lysis Buffer (Promega) according to manufacturers' instruction. Luminescence was detected using a Luminenscence GloMax™ Discover (Promega) with an integration time of 0.5 s.

Experiments were executed with both unlabeled and labeled (biotinylated) adalimumab as indicated. Controls included a B cell line expressing the B cell receptor for a known HIV-specific antibody (VRC01).

Study population: Patients were identified from the IBD clinic schedule, or the Vanderbilt GI endoscopy unit schedule based on screening by the study staff and clinical team. Control patients were identified from patients undergoing endoscopy for non-IBD indications. Potential subjects were contacted and invited to participate in the study prior to their scheduled endoscopy, in the IBD clinic, or when they are seen in the GI endoscopy unit.

The inclusion criteria were: (1) Patients who are able to give written informed consent; (2) Female or male patients ≥18 years old (3) Healthy volunteers without IBD being assessed in the GI Endoscopy Lab or patients with a diagnosis of IBD confirmed by endoscopy or radiology assessment being seen in the IBD clinic or the GI Endoscopy Lab for usual clinical care. This study had specific interest in IBD patients receiving adalimumab and not having clinically relevant anti-adalimumab antibodies, patients who stopped treatment with adalimumab due to clinically relevant anti-drug antibodies, and controls without IBD and without exposure to adalimumab. The exclusion criteria were: (1) Coagulopathy or bleeding disorder; (2) Renal or hepatic impairment; (3) History of organ transplantation including bone marrow transplantation; (4) Immunodeficiency; (5) Treatment with intravenous immunoglobulins; (5) Not suitable to participate in the study at discretion of clinical staff.

Data collection: Metadata including age, gender, height, weight, smoking status, and medication exposures as well as clinical indicators of disease activity and disease distribution were recorded in RedCap from the patient inclusion form and/or retrieved from the medical record by a physician.

Data safety: Blood samples were labeled with a study ID number and date of collection without direct patient identifiers. Only clinical staff and the PI on the IRB had access to the patient characteristics in RedCap.

Ethical considerations: Subjects provided written informed consent prior to any study-related procedure. This study was approved by the Vanderbilt University Medical Center Institutional Review Board, reference 200422.

Leukocyte and plasma collection: Blood was collected into a 10 mL EDTA tube and centrifuged at 800 g for 10 minutes at room temperature (22° C.) without brake. The plasma was spun again at 300 g for 8 minutes to remove any residual cells before being frozen in 1 mL aliquots. Leukocytes were then collected from the whole blood with Lympholyte™-h (Cedarlane Labs) and buffer (2% HI FBS with 2 mM EDTA in PBS) in a SepMate tube (Stem Cell Technologies) according to the manufacturers' instructions. Cells were frozen in liquid nitrogen in 90% HI FBS with 10% (v/v) DMSO until use. Cells were counted with a Cellometer Auto 2000 (Nexcelom).

Adalimumab and anti-adalimumab levels: Plasma adalimumab and anti-adalimumab levels at inclusion were determined by Sanquin (the Netherlands) as described before.¹⁰

Antigen titration: The variant amount was titrated using a normal control, a patient on adalimumab (without anti-drug antibodies) and a patient not on adalimumab with a high titre of anti-adalimumab antibodies using flow cytometry directed against CD19 (FITC or BV421), live/dead (AmCyan), and labeled adalimumab (PE).

Sequencing: For select experiments, samples were positively enriched for B cells using methods including negative selection (EasySep Human B cell Isolation Kit from Stem Cell Technologies) according to manufacturers' instruction.

Plasma TNF: Plasma TNF was assessed with a Human TNF alpha uncoated ELISA kit (Invitrogen) according to manufacturers' instruction.

Plasma IgG: Total plasma IgG was detected by a IgG (Total) Human Uncoated ELISA Kit (Invitrogen) according to manufacturers' instruction.

Analyses: Statistical analysis was done in Graphpad Prism. No formal power analysis was done as this was an exploratory study. A statistical analysis plan as well as data-driven analyses were made.

### Results

In silico variant design: For generating variants of adalimumab (hereafter variants), the 3.1 Angstrom co-crystal structure of the originator product adalimumab (hereafter adalimumab, brand name Humira) bound to TNF was used (PDDB ID 3WD5). Variants were designed by making an amino acid point mutation, i.e. choosing a single amino acid on adalimumab to be replaced by another amino acid. First, a pattern of (loss of) binding of anti-adalimumab antibodies to adalimumab was determined by analyzing the point mutations described previously.⁴ Then, in addition to non-surface epitopes residues that were solvent exposed and that covered new areas on the surface were chosen to help localize the epitopes recognized by B cells. Additionally, the study aimed to identify variants that had a lower global minimal energy conformation (GMEC) and lower free energy than either the originator product or the originator product bound to TNF. A “smart-search” algorithm was employed to identify low energy conformations of both adalimumab alone and adalimumab bound to TNF when mutations were introduced. Altogether, the study identified a number of variants (TABLE 1). As controls, an antibody against an irrelevant antigen (VRC01) as well as a variant with decreased binding to TNF (A50N_LC) were also made.

TABLE 1

| Chain | | | | % | | WT | Mutant | | | Mutant |
|---|---|---|---|---|---|---|---|---|---|---|
| and | Point | | | Increase | | Complex | Complex | | WT ab | ab |
| Region | Mutant | Interface? | Condition | in Kₐ | Condition | Stability | Stability | Condition | Stability | Stability |
| HCDR1 | T28H | No | NA | NA | 8 res, BB flex, | −44.96 | −51.23 | 6 res, no BB | 1.83 × 10³⁰ | 1.35 × 10³⁶ |
| | | | | | EPIC, GMEC | | | flex, Partition | | |
| | | | | | | | | Fn | | |
| HCDR1 | D31Q | No | NA | NA | 9 res, BB flex, | −58.36 | −62.41 | 6 res, no BB | 3.62 × 10³³ | 8.21 × 10³⁵ |
| | | | | | GMEC | | | flex, Partition | | |
| | | | | | | | | Fn | | |
| HCDR2 | W53K | No | NA | NA | 10 res BB flex, | −53.36 | −52.17 | 7 res, BB flex, | 1.55 × 10³¹ | 2.1 × 10³⁰ |
| | | | | | GMEC | | | Partition Fn | | |
| HCDR2 | N54H | No | NA | NA | 8 res, BB flex, | −49.31 | −50.18 | NA | NA | NA |
| | | | | | GMEC | | | | | |
| HCDR2 | S55G | No | NA | NA | 9 res, BB flex, | −55.88 | −63.32 | NA | NA | NA |
| | | | | | GMEC | | | | | |
| HCDR2 | G56Y | Yes | 6 res BB | 214% | 6 res, BB flex, | 2.80 × 10²⁶ | 2.20 × 10²⁸ | 6 res, BB flex, | 7.81 × 10¹⁹ | 2.88 × 10²¹ |
| | | | Flex, K* | | Partition Fn | | | Partition Fn | | |
| HCDR2 | H57W | Yes | 6 res BB | 20.10% | 6 res, BB flex, | 1.22 × 10³⁶ | 4.42 × 10³⁵ | 6 res, BB flex, | 1.52 × 10²⁵ | 2.74 × 10²⁵ |
| | | | Flex, K* | | Partition Fn | | | Partition Fn | | |
| HFR3 | D62K | No | NA | NA | 8 res, BB flex, | 7.075 | 3.373 | NA | NA | NA |
| | | | | | GMEC | | | | | |
| HCDR3 | Y101H | Yes | 6 res BB | 36.60% | 6 res, BB flex, | 2.40 × 10⁴⁰ | 1.77 × 10³⁹ | 6 res, BB flex, | 1.21 × 10¹⁹ | 2.47 × 10¹⁸ |
| | | | Flex, K* | | Partition Fn | | | Partition Fn | | |
| HCDR3 | L102K | Yes | NA | NA | 10 res, BB | −60.03 | −64.97 | NA | NA | NA |
| | | | | | flex, EPIC, | | | | | |
| | | | | | GMEC | | | | | |
| HCDR3 | S103W | Yes | 8 res, | 24066% | 8 res, BB Flex, | 6.4 × 10⁴⁸ | 2.75 × 10⁵³ | 8 res, BB flex, | 6.23 × 10¹⁹ | 1.11 × 10²² |
| | | | BB Flex, | | Partition Fn | | | Partition Fn | | |
| | | | K* | | | | | | | |
| HCDR3 | S103H | Yes | 8 res, | 138486% | 8 res, BB Flex, | 6.4 × 10⁴⁸ | 1.14 × 10⁵³ | 8 res, BB flex, | 6.23 × 10¹⁹ | 7.99 × 10²⁰ |
| | | | BB Flex, | | Partition Fn | | | Partition Fn | | |
| | | | K* | | | | | | | |
| HCDR3 | A105R | Yes | 8res, no | 582%* | 8 res, no BB | 2.98 × 10⁴⁴ | 2.69 × 10⁴⁴ | 8 res, no BB | 9.52 × 10²¹ | 1.47 × 10²¹ |
| | | | BB flex, | (Issue in | flex, Partition | | | flex, Partition | | |
| | | | K* | notes) | Fn | | | Fn | | |
| LCDR1 | Q27W | Yes | 8 res, | 147% | 8 res, BB Flex, | 8.82 × 10³⁹ | 3.81 × 10⁴⁰ | 8 res, BB | 3 × 10³² | 8.87 × 10³² |
| | | | BB flex, | | Partition Fn | | | Flex, Partition | | |
| | | | K* | | | | | Fn | | |
| LCDR1 | Q27R | Yes | 8 res, | 885% | 8 res, BB Flex, | 8.82 × 10³⁹ | 3.27 × 10³⁹ | 8 res, BB | 3 × 10³² | 1.25 × 10³⁹ |
| | | | BB flex, | | Partition Fn | | | Flex, Partition | | |
| | | | K* | | | | | Fn | | |
| LCDR1 | N31H | Yes | NA | NA | NA | NA | NA | NA | NA | NA |
| LCDR1 | N31R | Yes | 8 res, | 12% | 8 res, BB Flex, | 1.91 × 10³⁵ | 2.57 × 10³⁵ | 8 res, BB flex, | 2.27 × 10²² | 2.58 × 10²³ |
| | | | BB Flex, | | Partition Fn | | | Partition Fn | | |
| | | | K* | | | | | | | |
| LCDR2 | A50N | Yes | 8 res, | (1.29 × | 8 res, BB flex, | 7.54 × 10⁵² | 1.13 × 10⁴¹ | 8 res, BB flex, | 1.78 × 10³⁶ | 2.1 × 10³⁶ |
| | | | BB flex, | 10⁻¹⁰) % | LUTE, | | | LUTE, | | |
| | | | LUTE, | | Partition Fn | | | Partition Fn | | |
| | | | K* | | | | | | | |
| LFR3 | S60Y | No | NA | NA | 7 res, BB flex, | −56.67 | −58.67 | NA | NA | NA |
| | | | | | GMEC | | | | | |
| LFR3 | S67W | No | NA | NA | 10 res, BB | −51.63 | −54.62 | 6 res, no BB | 9.85 × 10²⁵ | 5.81 × 10²⁷ |
| | | | | | flex, GMEC | | | flex, Partition | | |
| | | | | | | | | Fn | | |
| LFR3 | S67E | No | NA | NA | 10 res, BB | −51.63 | −54.02 | 6 res, no BB | 9.85 × 10²⁵ | 4.92 × 10²⁸ |
| | | | | | flex, GMEC | | | flex, Partition | | |
| | | | | | | | | Fn | | |
| LFR3 | T69R | No | NA | NA | 9 res, BB flex, | −50.06 | −52.91 | NA | NA | NA |
| | | | | | GMEC | | | | | |
| LCDR3 | N92G | Yes | NA | NA | 8 res, BB flex, | 2539.22 | −7.41 | 6 res, BB flex, | 187.09 | −39.11 |
| | | | | | GMEC | | | GMEC | | |
| LCDR3 | A94Y | Yes | 11 res, | 6877% | 11 res, BB | 3.77 × 10⁸⁷ | 4.47 × 10⁹¹ | 11 res, BB | 7.18 × 10⁵⁶ | 1.24 × 10⁵⁹ |
| | | | BB flex, | | flex, LUTE, | | | flex, LUTE, | | |
| | | | LUTE, | | Partition Fn | | | Partition Fn | | |
| | | | K* | | | | | | | |

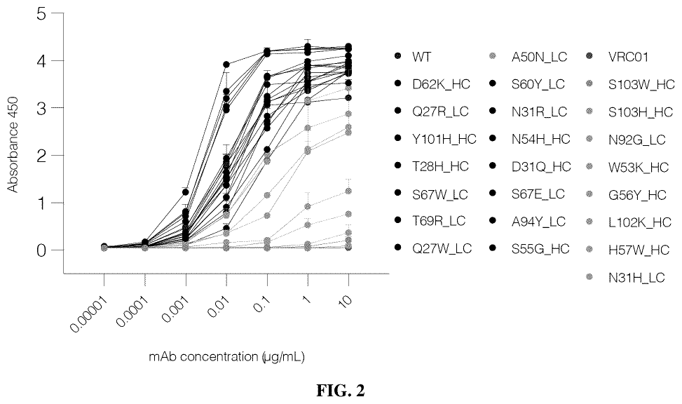

TNF binding ELISA: After generation of the variants, quality control was done as described in the methods. Then, an ELISA was done to determine the binding of the variants to TNF (FIG. 2). The data shows that, besides the controls, most variants bound to TNF with similar kinetics as unmodified adalimumab. Some variants showed decreased binding to TNF compared to unmodified adalimumab. Overall, the labeled variants had slightly decreased binding to TNF compared to the unlabeled variants.

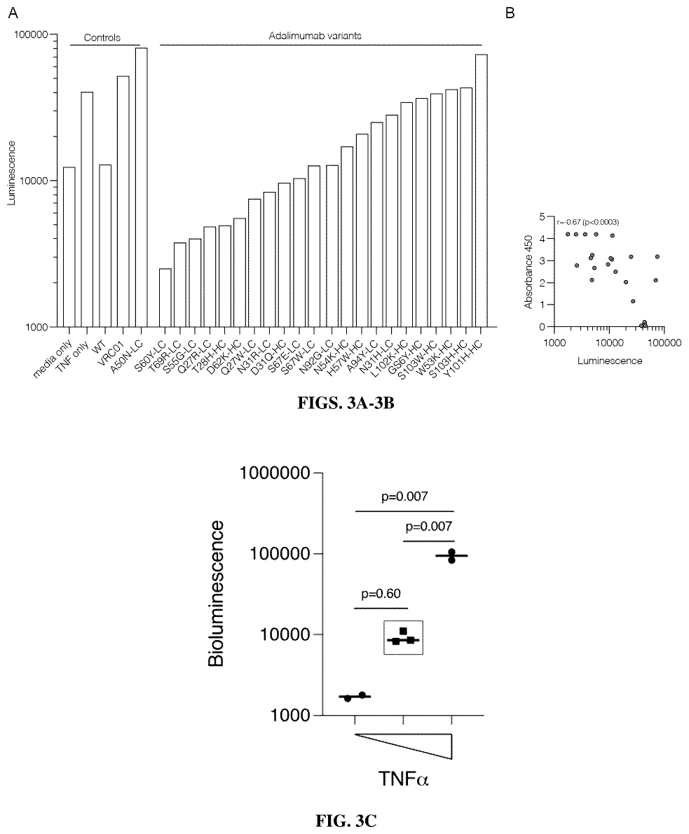

TNF neutralization: The TNF binding ELISA shows whether variants can bind to TNF but this is not necessarily the same as neutralizing the effect of TNF. To assess whether variants could neutralize the effect of TNF in vitro, the study used a stably transfected AGS cell line with an NF-KB luciferase reporter. The data shows that about half of the variants were better in neutralizing TNF in vitro compared to unmodified adalimumab and the other half was worse (FIG. 3A). The amount of binding positively correlated with the amount of TNF neutralization (FIGS. 3B-3C).

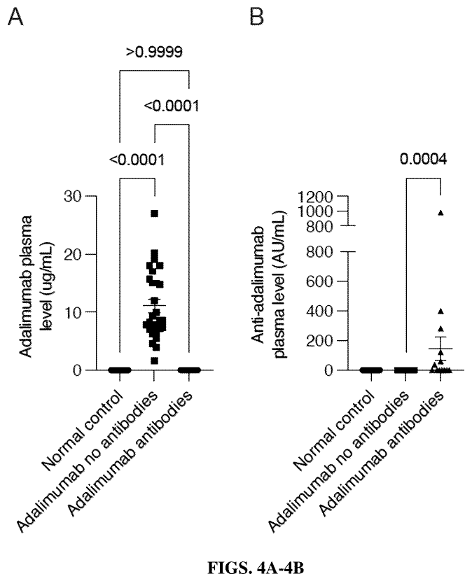

Clinical cohort: To assess to what epitopes of adalimumab patients develop anti-drug antibodies, the study collected leukocytes from healthy controls (hereafter controls), patients with inflammatory bowel disease (IBD) on adalimumab without clinically relevant anti-adalimumab antibodies (hereafter patients without anti-drug antibodies), and patients not on adalimumab with a history of drug-neutralizing antibodies. The study could not include patients on adalimumab with drug-neutralizing antibodies as it was expected that the presence of adalimumab would interfere with the labeling of antigen-specific B cells with adalimumab and variants. At inclusion adalimumab levels and anti-adalimumab antibody levels were determined. Only the patients on adalimumab at inclusion had detectable adalimumab levels (FIG. 4A). Anti-adalimumab antibody levels were then determined, and it was found that approximately half of the patients (N=6) with a history of drug-neutralizing anti-drug antibodies had anti-adalimumab antibodies at inclusion (hereafter patients with anti-drug antibodies, see FIG. 4B). The study prioritized sequencing the adalimumab specific B cells of these patients going forward.

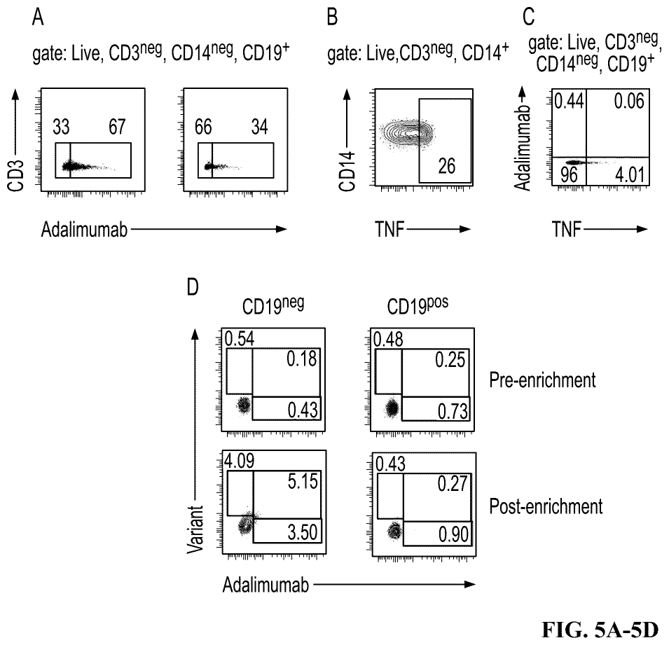

Sequencing of adalimumab specific B cells: Initially, the antigen positive B cells (Live CD14^{neg}CD3^{neg}CD19⁺Antigen⁺) of one patient and one healthy control were sequenced (FIG. 5A). Surprisingly, a higher fraction (as well as absolute number) of antigen-specific B cells were retrieved from the healthy control compared to the patient with anti-drug antibodies (FIG. 5A). The study focused its analysis on the sequencing from the patient. Interestingly, it was found that multiple variants were bound to multiple cells. However, some variants, including unmodified adalimumab, dropped out. Unmodified adalimumab dropping out was unexpected and indicated a technical problem with the method and therefore the study compared the TNF-binding of labeled adalimumab and variants to TNF as described above and made new adalimumab and variants where appropriate. Intriguingly, the data also indicated that adalimumab-specific B cells circulate long after adalimumab was stopped. Why the majority of the antigen-specific B cells was IgD and not IgG positive in this patient remains to be determined.

The study next assessed whether blocking with Fc-block would alter the detection of antigen-specific B cells in a patient with anti-drug antibodies. Surprisingly, blocking with Fc-block did not appreciably change the detection of antigen-specific B cells (data not shown), suggesting that adding Fc-block would not be a useful addition to the protocol. The study then assessed whether other cells would bind to the model antigen and therefore assessed whether monocytes (FIG. 5) or B cells (FIG. 5C) would express membrane-bound TNF using flow cytometry. Few antigen positive B cells expressed membrane bound TNF. However, a majority of monocytes (CD14⁺) expressed membrane-bound TNF. Altogether, this data shows that monocytes might act as a sink for adalimumab.

In an effort to optimize the method to detect more antigen-specific B cells, the study used negative immunomagnetic selection to enrich for CD19⁺ B cells. Despite the enrichment being successful, the remaining CD19^{neg} cells were still binding to antigen (FIG. 5D). Depleting the monocytes using positive selection followed by enrichment for B cells using negative selection may yield more antigen-specific B cells.

The following patents, applications and publications as listed below and throughout this document are hereby incorporated by reference in their entirety herein.

### REFERENCE LIST

1. Vermeire, S., Gils, A., Accossato, P., Lula, S., and Marren, A. (2018). Immunogenicity of biologics in inflammatory bowel disease. Therap Adv Gastroenterol 11, 1756283X1775035. 10.1177/1756283X17750355.
2. Baert, F., Kondragunta, V., Lockton, S., Casteele, N. vande, Hauenstein, S., Singh, S., Karmiris, K., Ferrante, M., Gils, A., and Vermeire, S. (2016). Antibodies to adalimumab are associated with future inflammation in Crohnis patients receiving maintenance adalimumab therapy: a post hoc analysis of the Karmiris trial. Gut 65, 1126-1131. 10.1136/gutjn1-2014-307882.
3. van Schouwenburg, P. A., van de Stadt, L. A., de Jong, R. N., van Buren, E. E. L., Kruithof, S., de Groot, E., Hart, M., van Ham, S. M., Rispens, T., Aarden, L., et al. (2013). Adalimumab elicits a restricted anti-idiotypic antibody response in autoimmune patients resulting in functional neutralisation. Ann Rheum Dis 72, 104-109. 10.1136/annrheumdis-2012-201445.
4. van Schouwenburg, P. A., Kruithof, S., Votsmeier, C., van Schie, K., Hart, M. H., de Jong, R. N., van Buren, E. E. L., van Ham, M., Aarden, L., Wolbink, G., et al. (2014). Functional analysis of the anti-adalimumab response using patient-derived monoclonal antibodies. Journal of Biological Chemistry 289, 34482-34488. 10.1074/jbc.M114.615500.
5. van Schie, K. A., Kruithof, S., van Schouwenburg, P. A., Vennegoor, A., Killestein, J., Wolbink, G., and Rispens, T. (2017). Neutralizing capacity of monoclonal and polyclonal anti-natalizumab antibodies: The immune response to antibody therapeutics preferentially targets the antigen-binding site. Journal of Allergy and Clinical Immunology 139, 1035-1037.e6. 10.1016/j.jaci.2016.09.014.
6. van Schie, K. A., Hart, M. H., de Groot, E. R., Kruithof, S., Aarden, L. A., Wolbink, G. J., and Rispens, T. (2015). The antibody response against human and chimeric anti-TNF therapeutic antibodies primarily targets the TNF binding region. Ann Rheum Dis 74, 311-314. 10.1136/annrheumdis-2014-206237.
7. Setliff, I., McDonnell, W. J., Raju, N., Bombardi, R. G., Murji, A. A., Scheepers, C., Ziki, R., Mynhardt, C., Shepherd, B. E., Mamchak, A. A., et al. (2018). Multi-Donor Longitudinal Antibody Repertoire Sequencing Reveals the Existence of Public Antibody Clonotypes in HIV-1 Infection. Cell Host Microbe 23, 845-854.e6. 10.1016/j.chom.2018.05.001.
8. Setliff, I., Shiakolas, A. R., Pilewski, K. A., Acharya, P., Morris, L., and Georgiev, I. S. (2019). High-Throughput Mapping of B Cell Receptor Sequences to Antigen Specificity Resource High-Throughput Mapping of B Cell Receptor Sequences to Antigen Specificity. Cell 179, 1-11. 10.1016/j.cell.2019.11.003.
9. Hu, S., Liang, S., Guo, H., Zhang, D., Li, H., Wang, X., Yang, W., Qian, W., Hou, S., Wang, H., et al. (2013). Comparison of the inhibition mechanisms of Adalimumab and Infliximab in treating tumor necrosis factor α-associated diseases from a molecular view. Journal of Biological Chemistry 288, 27059-27067. 10.1074/jbc.M113.491530. Jacobse, J., Voorde, W., Tandon, A., Romeijn, S. G., Grievink, H. W., Maaden, K., Esdonk,
10. M. J., Moes, D. J. A. R., Loeff, F., Bloem, K., et al. (2021). Comprehensive evaluation of microneedle—based intradermal adalimumab delivery vs subcutaneous administration: results of a randomized controlled clinical trial. Br J Clin Pharmacol 87, 3162-3176. 10.1111/bcp.14729.

SEQUENCES

Adalimumab_HC
EVQLVESGGGLVQPGRSLRLSCAASGFTFDDYAMHWVRQAPGKGLEWVSAITWNSGHI
DYADSVEGRFTISRDNAKNSLYLQMNSLRAEDTAVYYCAKVSYLSTASSLDYWGQGTL
VTVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSC (SEQ ID NO: 1)
Adalimumab_LC
DIQMTQSPSSLSASVGDRVTITCRASQGIRNYLAWYQQKPGKAPKLLIYAASTLQSGVPS
RFSGSGSGTDFTLTISSLQPEDVATYYCQRYNRAPYTFGQGTKVEIKRTVAAPSVFIFPPS
DEQLKSGTASVVCLLNNFYPREAKVQWKVDNALQSGNSQESVTEQDSKDSTYSLSSTL
TLSKADYEKHKVYACEVTHQGLSSPVTKSFNRGEC (SEQ ID NO: 2)

What is claimed is:

1. A method for generating deimmunized antibody therapeutics, comprising:
labeling a plurality of mutated variants of an antibody therapeutic with unique barcodes;
providing a plurality of barcode-labeled mutated variants to a population of antibody therapeutic-specific B-cells;
allowing the plurality of barcode-labeled mutated variants to bind to the population of antibody therapeutic-specific B-cells; and
identifying unbound mutated variants as having lower immunogenicity than the antibody therapeutic.

2. The method of claim 1, further comprising:
determining, based on mutations present in bound and unbound mutated variants, an epitope of the antibody therapeutic; and
preparing one or more additional mutated variants having lower immunogenicity than the antibody therapeutic based on the epitope of the antibody therapeutic.

3. The method of claim 1, wherein the unique barcodes comprise DNA sequences or RNA sequences.

4. The method of claim 1, wherein the antibody therapeutic comprises adalimumab.

5. The method of claim 1, wherein the population of antibody therapeutic-specific B-cells comprises a memory B-cell, a plasma cell, a naïve B cell, an activated B-cell, or a B-cell line.

6. The method of claim 5, wherein the population of antibody therapeutic-specific B cells is derived from a patient.

7. The method of claim 1, wherein the mutated variant comprises one or more point mutations.

8. The method of claim 1, further comprising:
assessing binding to and/or neutralization of a target antigen by the mutated variants.

9. The method of claim 8, wherein the target antigen is tumor necrotic factor alpha (TNFα).

10. The method of claim 1, further comprising:
preparing a modified antibody therapeutic comprising one or more mutated variants having lower immunogenicity than the antibody therapeutic.

11. The method of claim 10, further comprising:
administering the modified antibody therapeutic to a patient in need thereof;
wherein, when administered to the patient, the modified antibody therapeutic generates less anti-drug antibodies than the antibody therapeutic without modifications.

12. A system for generating deimmunized antibody therapeutics, comprising:
a plurality of barcode-labeled mutated variants of an antibody therapeutic;
a population of antibody therapeutic-specific B-cells; and
wherein the mutated variants are identified by a non-transitory computer-readable medium having instructions stored thereon, wherein the instructions, when executed by a processor, cause the processor to:
determine mutations and/or combinations of mutations to the antibody therapeutic which do not substantially interfere with binding of the mutated variant to a target antigen.

13. The system of claim 12, wherein the instructions, when executed by the process, further cause the process to determine mutations and/or combinations of mutations to the antibody therapeutic which minimize the possibility of interaction with the population of antibody-specific B-cells.

14. The system of claim 12, wherein barcodes comprise DNA sequence or RNA sequences.

15. The system of claim 12, wherein the target antigen is tumor necrotic factor alpha (TNFα).

16. The system of claim 15, wherein the antibody therapeutic comprises adalimumab.

17. The system of claim 12, wherein the population of antibody therapeutic-specific B-cells comprises a memory B-cell, a plasma cell, a naïve B cell, an activated B-cell, or a B-cell line.

18. The system of claim 17, wherein the population of antibody therapeutic-specific B cells is derived from a patient.

19. A mutated variant of adalimumab, comprising one or more mutations to SEQ ID NO: 1 and/or one or more mutations to SEQ ID NO: 2;
wherein the one or more mutations to SEQ ID NO: 1 are selected from the group consisting of D62K, Y101H, T28H, N54H, N54K, D31Q, S55G, S103W, S103H, W53K, G56Y, L102K, and H57W; or
wherein the one or more mutations to SEQ ID NO: 2 are selected from the group consisting of Q27R, T69R, Q27W, A50N, S60Y, N31R, S67E, S67W, A94Y, N92G, and N31H.


SEQUENCE LISTING (2 sequences)

SEQ ID NO: 1 - protein, 224 aa, synthetic construct
  REGION 1..224: note = Description of sequence: heavy chain of adalimumab
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 2 - protein, 214 aa, synthetic construct
  REGION 1..214: note = Description of sequence: light chain of adalimumab
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC